(19) United States
(12) Patent Application Publication
(10) Pub. No.: US 2018/0201990 A1
(43) Pub. Date: Jul. 19, 2018

(54) Kit, apparatus, and method for detecting chromosome aneuploidy

(71) Applicants: Nanjing Annoroad Gene Technology Co., Ltd., Nanjing (CN); Annoroad Gene Technology (Beijing) Co., Ltd., Beijing (CN)

(72) Inventors: Chongjian CHEN, Beijing (CN); Junbin LIANG, Beijing (CN); Zhaoling XUAN, Beijing (CN); Dawei LI, Beijing (CN)

(21) Appl. No.: 15/571,859

(22) PCT Filed: May 6, 2015
(86) PCT No.: PCT/CN2015/078422

§ 371 Date: Nov. 6, 2017

(51) Int. Cl. C12Q 1/6869; G06F 19/18; G06F 19/12; G06F 19/22; C12Q 1/6883; C12Q 1/6827

(52) U.S. Cl. CPC C12Q 1/6869; G06F 19/18; C12Q 1/6827; G06F 19/22; C12Q 1/6883; G06F 19/12

(57) ABSTRACT

A kit, an apparatus, and a method for detecting chromosome aneuploidy. The method comprises: sequencing the peripheral blood cell-free DNA of a pregnant woman to be tested to produce sequencing data comprising all chromosomes; calculating a coverage for all of the chromosomes in the sequencing data by segmenting the chromosomes into windows so as to produce a pre-correction coverage for the each chromosome; calculating a Z_{CNV} value using the number of unique sequences in each window and producing fragments with copy number variation of the pregnant woman on the basis of the magnitude of the Z_{CNV} value; by utilizing the impact that the fragments with copy number variation have on the pre-correction coverage, correcting the pre-correction coverage to produce a corrected coverage; calculating a Zₐₙₑᵤ value for the each chromosome by utilizing the corrected coverage of the each chromosome; and, if the absolute value of the Zₐₙₑᵤ value is greater than or equal to 3, then it is determined that the chromosome has an aneuploidy.

### FIELD OF THE INVENTION

The present invention relates to the biomedical field, more particularly, to a kit, an apparatus and a method for detecting chromosome aneuploidy.

### BACKGROUND OF THE INVENTION

It has been 20 years since cell-free fetal DNA (cff-DNA) was found by Lo in 1997, which has provided possibility for varieties of non-invasive prenatal testing (NIPT). NIPT is advantageous in two aspects: on the one hand, NIPT will not cause any miscarriage risk, but the invasive manners including amniocentesis and cordocentesis for chromosome karyotype analysis will bring about 1/200 miscarriage risk, and there were researches indicating that cordocentesis may also cause fetus position tilted; on the other hand, NIPT can be performed as early as the 8th week of pregnancy, which provides earlier risk evaluation so as to decrease needs of induced labour for pregnant women.

These advantages lead to NIPT relevant methods developing rapidly and being widely applied as well. In current, there have been NIPT for fetal chromosome aneuploidy detection, NIPT for fetal single gene diseases, NIPT for fragment with copy number variation (CNV) in fetus, NIPT for fetal whole genome, NIPT for fetal paternity test and the like.

At present, among all of the NIPT applications, the most widely used and developed one is the fetal chromosome aneuploidy detection. In a number of methods for fetal chromosome aneuploidy detection, Chui's invention based on massively parallel sequencing (MPS) in 2008 is considered to be the most suitable one for clinical use, which has already exhibited its robustness. For Down syndrome, the false positive rate (FPR) can reach up to 0.443%, and the false negative rate (FNR) is as low as 0.004%; for Edward's syndrome, the FPR is 0.22%, and the FNR is 0.025%.

Although above methods had achieved such low error rates, risks based on wrong judgments still exist. Therefore, improvement for existing methods is in demand so as to decrease the error rate of detection as low as possible.

### SUMMARY OF THE INVENTION

The main object of the present application is to provide a kit, an apparatus and a method for detecting chromosome aneuploidy so as to reduce the false positive rate of the detection.

In order to achieve above object, according to one aspect of the present application, provided is a method for detecting chromosome aneuploidy, which includes the following steps of: high-throughput sequencing of the peripheral blood cell-free DNA from a pregnant woman to be tested to produce sequencing data comprising all of the chromosomes;

calculating coverage statistics for all of the chromosomes with the sequencing data by segmenting the chromosomes into windows so as to produce a pre-correction coverage for each chromosome;

performing a Z-test on the number of unique sequence in each window of the pregnant woman to produce a Z_{CNV} value and then locating chromosomal fragment with the copy number variation of the pregnant woman on the basis of the magnitude of the Z_{CNV} value; wherein chromosomal fragment with the copy number variation of the pregnant woman is the one which is 300 Kb or more in length and which has the Z_{CNV} values of the chromosome fragments greater than or equal to 4 or less than or equal to −4 in 80% or more of the total windows within the fragment which is 300 Kb or more in length,

correcting the pre-correction coverage of the each chromosome by utilizing the impact of the fragment with copy number variation of the pregnant woman on the pre-correction coverage of the each chromosome to produce the corrected coverage for the each chromosome; and

performing a Z-test for the each chromosome by using the corrected coverage of the each chromosome to obtain the Zₐₙₑᵤ value, and determining whether the chromosome has an aneuploidy based on whether the absolute value of Zₐₙₑᵤ is greater than or equal to 3; wherein when the absolute value of Zₐₙₑᵤ is greater than or equal to 3, then it is determined that the chromosome has an aneuploidy;

wherein the impact of the fragment with copy number variation of the pregnant woman to be tested on the pre-correction coverage of the each chromosome is represented by a parameter α,

when the fetus inherits the fragment with copy number variation from the mother, the parameter α is calculated as formula (1):

$$α = \frac{( m - n ) \cdot 2 + n \cdot cn}{m \cdot 2} \qquad ( 1 )$$

when the fetus does not inherit the fragment with copy number variation from the mother, the parameter α is calculated as formula (2):

$$α = \frac{( m - n ) \cdot 2 + f \cdot n \cdot 2 + ( 1 - f ) \cdot n \cdot cn}{m \cdot 2} \qquad ( 2 )$$

in formula (1) and formula (2), m represents the effective length of the chromosome in which the fragment with copy number variation occurs, in the unit of Mb; and n represents the length of the fragment with copy number variation of the pregnant woman to be tested, in the unit of Mb; cn represents the copy number of the fragment with copy number variation found in the pregnant woman to be tested;

in formula (2), f represents the concentration of the cell-free fetal DNA existing in the peripheral blood cell-free DNA of the pregnant woman to be tested, and the concentration f of the cell-free fetal DNA is assumed to be less than 50%;

correcting the pre-correction coverage of the each chromosome by using

$${x}^{′} = \frac{\hat{x}}{α} ,$$

wherein \hat{x} represents the pre-correction coverage of the each chromosome and x′ represents the corrected coverage of the each chromosome.

Furthermore, the coverage statistics is calculated by segmenting all of the chromosomes in the sequencing data into windows with equal sizes so as to produce the pre-correction coverage of the each chromosome.

In addition, the length of the each window is 100 Kb, and the overlapping ratio between two adjacent windows is 50%.

Further, the step of performing a Z-test on the number of unique sequences in the each window of the pregnant woman to be tested to produce the Z_{CNV} value and then locating chromosomal fragment with the copy number variation of the pregnant woman to be tested on the basis of the magnitude of the Z_{CNV} value further includes the steps of:

counting the number of the unique sequences in the each window according to the sequencing depth of each sequence in the sequencing data;

calculating the number of the unique sequences in the each window according to the GC content and the mapping rate of the each chromosome to obtain the pre-correction coverage of the number of the unique sequences in the each window; and

normalizing the pre-correction coverage of the number of the unique sequences in the each window to obtain the Z_{CNV} value of the number of the unique sequences in the each window and determining whether the pregnant woman to be tested has the chromosomal fragment with copy number variation on the basis of the magnitude of the Z_{CNV} value;

if there is a fragment which is 300 Kb or more in length in the sequencing data, and within the fragment which is 300 Kb or more in length, the Z_{CNV} values of the numbers of the unique sequences in 80% or more of the total windows are greater than or equal to 4 or less than or equal to −4, then the fragment which is 300 Kb or more in length is determined to be the fragment with copy number variation of the pregnant woman to be tested.

Moreover, for the step of performing a Z-test for the each chromosome by using the corrected coverage of the each chromosome to obtain the Zₐₙₑᵤ value, the Zₐₙₑᵤ value is calculated as:

$${Z}_{aneu} = \frac{{x}^{′} - \overline{x}}{s}$$

wherein x̅ represents the pre-correction coverage obtained by the known negative sample population according to a LOESS algorithm; s represents the standard error of (x′−x̅) in the negative sample population.

In order to achieve above object, in another aspect of the present application, provided is an apparatus for detecting chromosome aneuploidy, which includes the following modules:

a sequencing data detection module: for high-throughput sequencing the peripheral blood cell-free DNA of a pregnant woman to be tested to produce the sequencing data comprising all of the chromosomes;

a first coverage calculation module: for calculating coverage statistics of all of the chromosomes with the sequencing data by segmenting the chromosomes into windows so as to produce a pre-correction coverage for each chromosome;

a Z_{CNV} value calculation module: for calculating the Z_{CNV} value on the number of unique sequences in each window of the pregnant woman;

a fragment with copy number variation search module: for searching the fragment that is 300 Kb or more in length in the sequencing data and which has the Z_{CNV} values of the chromosome fragments greater than or equal to 4 or less than or equal to −4 in 80% or more of the total windows;

a fragment with copy number variation determination module: for determining a fragment in the sequencing data that is 300 Kb or more in length and which has Z_{CNV} values of the chromosome fragments greater than or equal to 4 or less than or equal to −4 in 80% or more of the total windows as the fragment with copy number variation of the pregnant woman;

a first α calculation module: for calculating the parameter α according to the formula (1) in the case where the fetus inherits the fragment with copy number variation from the mother, wherein the parameter α represents the impact of the fragment with copy number variation of the pregnant woman on the pre-correction coverage of the each chromosome; m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman;

$$α = \frac{( m - n ) \cdot 2 + n \cdot cn}{m \cdot 2} \qquad ( 1 )$$

a second α calculation module: for calculating the parameter α according to formula (2) in the case where the fetus does not inherit the fragment with copy number variation from the mother, wherein the parameter α is calculated according to formula (2),

$$α = \frac{( m - n ) \cdot 2 + f \cdot n \cdot 2 + ( 1 - f ) \cdot n \cdot cn}{m \cdot 2} \qquad ( 2 )$$

in formula (2), m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;

a correction module: for correcting the pre-correction coverage of the each chromosome by using:

$${x}^{′} = \frac{\hat{x}}{α} ,$$

to produce the corrected coverage of the each chromosome; wherein \hat{x} represents the pre-correction coverage of the each chromosome and x′ represents the corrected coverage of the each chromosome;

a second coverage calculation module: for calculating the Zₐₙₑᵤ value of the each chromosome by using the corrected coverage of the each chromosome;

Zₐₙₑᵤ value determination module: for determining whether the absolute Zₐₙₑᵤ value is greater than or equal to 3;

a chromosome aneuploidy confirmation module: for confirming the chromosome has aneuploidy in the case where the absolute Zₐₙₑᵤ value is greater than or equal to 3.

Further, the first coverage calculation module includes:

a chromosome window segmentation sub-module: for segmenting all of the chromosomes in the sequencing data into windows with equal size;

a first coverage calculation sub-module: for calculating the coverage statistics in the form of windows with equal size to produce the pre-correction coverage of the each chromosome.

In addition, the size of the each window in the chromosome window segmentation sub-module is 100 Kb, and the overlapping ratio between two adjacent windows is 50%.

Furthermore, the Z_{CNV} value calculation module includes:

a unique sequence counting unit: for counting the number of the unique sequences in the each window according to the sequencing depth of each sequence in the sequencing data;

a unique sequence coverage calculation unit: for calculating the number of the unique sequences in the each window according to the GC content and the mapping rate of the each chromosome to obtain the pre-correction coverage of the number of the unique sequences in the each window; and

a unique sequence Z_{CNV} value calculation unit: for normalizing the pre-correction coverage of the number of the unique sequences in the each window to obtain the Z_{CNV} value of the number of the unique sequences in the each window.

Additionally, in the second coverage calculation module, the Zₐₙₑᵤ value is calculated as:

$${Z}_{aneu} = \frac{{x}^{′} - \overline{x}}{s}$$

wherein x̅ is the pre-correction coverage obtained by the known negative sample population according to a LOESS algorithm; s represents the standard error of (x′−x̅) in the negative sample population.

According to another aspect of the present application, provided a kit for detecting chromosome aneuploidy, includes:

the detection reagents and a detection device: for high-throughput sequencing the peripheral blood cell-free DNA from a pregnant woman to be tested to produce the sequencing data containing all of the chromosomes;

a first coverage calculation device: for calculating coverage statistics of all of the chromosomes with the sequencing data by segmenting the chromosomes into windows so as to produce a pre-correction coverage for the each chromosome;

a Z_{CNV} value calculation device: for performing a Z-test on the number of unique sequences in each window from the pregnant woman to be tested to obtain the Z_{CNV} value;

a fragment with copy number variation search device: for searching the fragment in the sequencing data that is 300 Kb or more in length and which has the Z_{CNV} values of the chromosome fragments greater than or equal to 4 or less than or equal to −4 in 80% or more of the total windows;

a fragment with copy number variation determination device: for obtaining the fragment with copy number variation fragment of the pregnant woman to be tested on the basis of the magnitude of the Z_{CNV} value;

a first α calculation device: for calculating the parameter α according to the formula (1) in the case where the fetus inherits the fragment with copy number variation from the mother, wherein the parameter α represents the impact of the fragment with copy number variation of the pregnant woman to be tested on the pre-correction coverage of the each chromosome;

$$α = \frac{( m - n ) \cdot 2 + n \cdot cn}{m \cdot 2} \qquad ( 1 )$$

m represents the effective length of the chromosome in which the fragment with copy number variation occurs, in the unit of Mb; and n represents the length of the fragment with copy number variation of the pregnant woman to be tested, in the unit of Mb; cn represents the copy number of the fragment with copy number variation found in the pregnant woman;

a second α calculation device: for calculating the parameter α according to formula (2) in the case where the fetus does not inherit the fragment with copy number variation from the mother, wherein the parameter α is calculated according to formula (2)

$$α = \frac{( m - n ) \cdot 2 + f \cdot n \cdot 2 + ( 1 - f ) \cdot n \cdot cn}{m \cdot 2} \qquad ( 2 )$$

m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;

a correction device: for correcting the pre-correction coverage of the each chromosome by using:

$${x}^{′} = \frac{\hat{x}}{α} ,$$

to produce the corrected coverage of the each chromosome; wherein \hat{x} represents the pre-correction coverage of the each chromosome and x′ represents the corrected coverage of the each chromosome;

a second coverage calculation device: for calculating the Zₐₙₑᵤ value of the each chromosome by using the corrected coverage of the each chromosome;

Zₐₙₑᵤ value determination device: for determining whether the absolute Zₐₙₑᵤ value is greater than or equal to 3;

a chromosome aneuploidy confirmation device: for confirming the chromosome has aneuploidy in the case where the absolute Zₐₙₑᵤ value is greater than or equal to 3.

Further, the first coverage calculation device includes:

a chromosome window segmentation component: for segmenting all of the chromosomes in the sequencing data into windows with equal size;

a first coverage calculation component: for calculating the coverage statistics in the form of windows with equal size to produce the pre-correction coverage of the each chromosome.

Furthermore, the size of the each window in the chromosome window segmentation component is 100 Kb, and the overlapping ratio between two adjacent windows is 50%.

In addition, the Z_{CNV} value calculation device includes:

a unique sequence counting component: for counting the number of the unique sequences in the each window according to the sequencing depth of each sequence in the sequencing data;

a unique sequence coverage calculation component: for calculating the number of the unique sequences in the each window according to the GC content and the mapping rate of the each chromosome to obtain the pre-correction coverage of the number of the unique sequences in the each window; and

a unique sequence Z_{CNV} value calculation component: for normalizing the pre-correction coverage of the number of the unique sequences in the each window to obtain the Z_{CNV} value of the number of the unique sequences in the each window.

Additionally, in the second coverage calculation device, the Zₐₙₑᵤ value is calculated as:

$${Z}_{aneu} = \frac{{x}^{′} - \overline{x}}{s}$$

wherein x̅ is the pre-correction coverage obtained by the known negative sample population according to a LOESS algorithm; s represents the standard error of (x′−x̅) in the negative sample population.

According to the technical solution of the present application, by screening the fragment with copy number variation occurring on the chromosome of the mother, and by determining whether the chromosome has an aneuploidy based on removing the impact of the copy number variation on the coverage of each chromosome, thereby the corrected coverage of the each chromosome can be obtained. By utilizing the corrected coverage to calculate and determine the chromosome aneuploidy, the present application can achieve a more accurate result.

### DESCRIPTION OF FIGURES

The accompanying figures, which are incorporated in and constitute a part of this application, are intended to provide a further understanding of the present application, and the illustrative examples of the present application and the description thereof are intended to explain the present application, which should not be construed as limiting the scope of the present application. In the figures:

FIG. 1 shows a flow diagram of a method for detecting chromosome aneuploidy according to a typical embodiment of the present application;

FIG. 2 shows a schematic diagram of a apparatus for detecting chromosome aneuploidy according to a typical embodiment of the present application;

FIGS. 3A, 3B and 3C respectively shows the corrected results indicating aneuploidy detection of chromosome 13, chromosome 18 and chromosome 21 according to Example 1 of the present application;

FIG. 4 shows the corrected result indicating aneuploidy of samples EK01875 and BD01462 on chromosome 21 according to Example 2 of the present application;

FIG. 5 shows the corrected result indicating aneuploidy detection of sample EK01875 on chromosome 21 according to Example 3 of the present application; and

FIG. 6 shows the corrected result indicating aneuploidy detection of sample BD01462 on chromosome 21 according to Example 4 of the present application.

### DETAILED DESCRIPTION OF THE INVENTION

It is to be noted that the features in the embodiments and examples of the present application can be combined with each other in a non-conflicting way. Hereinafter, the present application will be described in detail with reference to the embodiments.

In this application. Z_{CNV} or Zₐₙₑᵤ refers to the statistic value calculated by the Z-test, a method for testing mean difference of samples with large size (i.e. the sample size is greater than 30). It applies standard normal distribution theory to analyze the probability of occurrence of differences so as to conclude whether the difference between two averages is significant.

Mapping rate refers to a ratio obtained by aligning the sequencing sequence within the window to the reference sequence in genome. Since the sequencing sequences in the windows may be aligned to multiple sites of the reference sequence in genome but not an unique sequence, the mapping rate in the window is larger than that of an unique sequence.

It is to be noted that by extensive analysis, the applicant of the present application has found that there are at least three possibilities causing misjudgments of NIPT through conventional methods:

First, Lo found that cff-DNA was derived from placenta in 1998, which means that if confined placental mosaicism (CPM) appears, it will be difficult to accurately estimate the situation of the fetus by NIPT and the results more likely to be inaccurate; second, if CNV exists in the pregnant woman herself, the method which is based on the MPS statistical coverage and is also converted to the Z value will be inaccurate. Therefore, when repeat fragments present in the pregnant woman, the relative numbers of unique sequences aligned to the chromosomes will increase, and the Z value will also increase as the increase of the coverage, thereby increasing the risk of false positives. Conversely, if there is a fragment deletion in the pregnant woman, the Z value will decrease, thereby increasing the risk of false negatives. Moreover, some of the previous studies have also shown that confined placental mosaicism (CPM) and copy number variation (CNV) are major reasons for false positive. Finally, during the calculation of the chromosome coverage and the correction of the coverage by GC content, there may be data fluctuation, thereby resulting in errors.

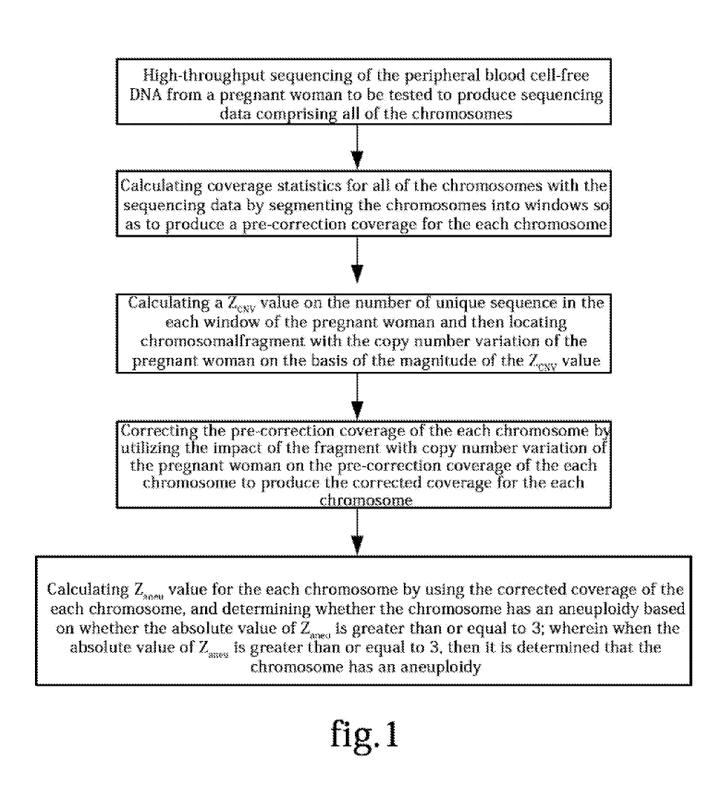

To this end, based on a comprehensive analysis for above-mentioned reasons for the errors, the present application proposes a method for detecting chromosome aneuploidy, as shown in FIG. 1, which includes the steps of:

high-throughput sequencing of the peripheral blood cell-free DNA from a pregnant woman to be tested to produce sequencing data comprising all of the chromosomes;

calculating coverage statistics for all of the chromosomes with the sequencing data by segmenting the chromosomes into windows so as to produce a pre-correction coverage for each chromosome;

calculating a Z_{CNV} value on the number of unique sequence in the each window of the pregnant woman and then locating the fragment with copy number variation of the pregnant woman on the basis of the magnitude of the Z_{CNV} value; wherein the chromosomal fragment with copy number variation of the pregnant woman is the one which is 300 Kb or more in length in the sequencing data and has the Z_{CNV} values of the chromosome fragments greater than or equal to 4 or less than or equal to −4 in 80% or more of the total windows among the fragment which is 300 Kb or more in length,

correcting the pre-correction coverage of the each chromosome by utilizing the impact of the fragment with copy number variation of the pregnant woman on the pre-correction coverage of the each chromosome to produce the corrected coverage for the each chromosome; and

using the corrected coverage of the each chromosome to obtain the Zₐₙₑᵤ value for the each chromosome, and determining whether the chromosome has an aneuploidy based on whether the absolute value of Zₐₙₑᵤ is greater than or equal to 3; wherein when the absolute value of Zₐₙₑᵤ is greater than or equal to 3, then it is determined that the chromosome has an aneuploidy;

wherein the impact of the fragment with copy number variation of the pregnant woman to be tested on the pre-correction coverage of the each chromosome is represented by a parameter α,

when the fetus inherits the fragment with copy number variation from the mother, the parameter α is calculated as formula (1):

$$α = \frac{( m - n ) \cdot 2 + n \cdot cn}{m \cdot 2} \qquad ( 1 )$$

when the fetus does not inherit the fragment with copy number variation from the mother, the parameter α is calculated as formula (2):

$$α = \frac{( m - n ) \cdot 2 + f \cdot n \cdot 2 + ( 1 - f ) \cdot n \cdot cn}{m \cdot 2} \qquad ( 2 )$$

in formula (1) and formula (2), m represents the effective length of the chromosome in which the fragment with copy number variation occurs, in the unit of Mb; and n represents the length of the fragment with copy number variation of the pregnant woman to be tested, in the unit of Mb; cn represents the copy number of the fragment with copy number variation found in the pregnant woman to be tested;

in formula (2), f represents the concentration of the cell-free fetal DNA existing in the peripheral blood cell-free DNA of the pregnant woman to be tested, and the concentration f of the cell-free fetal DNA is assumed to be less than 50%; and

correcting the pre-correction coverage of the each chromosome by using

$${x}^{′} = \frac{\hat{x}}{α} ,$$

wherein \hat{x} represents the pre-correction coverage of the each chromosome and x′ represents the corrected coverage of the each chromosome.

In prior art, the fragment with copy number variation of the mother in the sequencing data will be removed directly without further consideration, however, The present application is not the same as the prior art, the method of the present application screens the fragment with copy number variation with certain length occurring on the chromosome of the mother, and the impact of the fragment with copy number variation on calculating the coverage of each chromosome is further removed while determining the aneuploidy of the chromosome, thereby obtaining a corrected coverage for each chromosome so as to achieve a more accurate result for chromosome aneuploidy according to the method of the present application.

In above method of the present application, the method for calculating the concentration f of cell-free fetal DNA contained in the peripheral blood cell-free DNA of the pregnant woman is a conventional calculation method in the art. For example, when the fetus is male, and when the fragment with copy number variation is in the X chromosome, the concentration of cell-free fetal DNA is calculated according to

$$f = 2 ( 1 - \frac{{\overline{N}}_{23}}{\overline{N}} )$$

wherein

$$\frac{{\overline{N}}_{23}}{\overline{N}}$$

represents the average number of the unique sequences in windows on the X chromosome to the average number of the unique sequences in all of the windows; and when the fragment with copy number variation occurs on chromosome 21, 18 or 13, the concentration of the cell-free fetal DNA is calculated as

$$f = 2 ( \frac{{\overline{N}}_{i}}{\overline{N}} - 1 ) :$$

wherein

$$\frac{{\overline{N}}_{i}}{\overline{N}}$$

represents ratio of the average number of the unique sequences in windows on the chromosome 21, 18 or 13 to the average number of the unique sequences in all of the windows. When the fetus is female, specific gene methylation detection for the peripheral blood cell-free DNA of the pregnant woman is needed. The principle is that certain genes have different forms of methylation in the DNA of the pregnant woman from the DNA of the fetus. For example, RASSF1A gene (on chromosome 3) from the fetus and the placental origins is highly methylated, however, RASSF1A gene from the mother herself is unmethylated. By treating the cffDNA by using methylation sensitive enzymes such as HhaI, BstUI (30 U) and HpaII, unmethylated gene will be digested and the methylated gene will not be digested, by which the content of fetal cffDNA can be detected through Q-PCR. The specific procedures can reference PLOS ONE 9: 71-7 (2014), Quantification of Cell-Free DNA in Normal and Complicated Pregnancies: Overcoming Biological and Technical Issues.

In the above-described method of the present application, while calculating the pre-correction coverage of the each chromosome, because the chromosome is segmented into windows for calculation, relatively robust chromosome coverage can be obtained. Thus, in a preferred embodiment of the present application, the coverage statistics is calculated by segmenting all of the chromosomes in the sequencing data into windows with equal sizes so as to produce the pre-correction coverage of the each chromosome.

In a more preferred embodiment of the present application, during process of the calculation of coverage by segmenting into windows, the length of the each window is 100 Kb and the overlapping ratio between two adjacent windows is 50%. By controlling the length of the each window as 100 Kb and the ratio of overlap between the two adjacent windows as 50%, one cannot only obtain a relatively more robust chromosome coverage, but can also increase the accuracy for the detection of the fragment with copy number variation through the increased overlapping ratio between windows so as to increase the detection efficiency of the fragment with copy number variation of the pregnant woman.

In the above-described method of the present application, based on the procedures of conventional methods for calculating the fragment with copy number variation, and according to the difference of the qualities of the sequencing data or accuracies of detections, it can be obtained by appropriately adjusting the condition met by the fragment with copy number variation. In a preferred embodiment of the present application, calculating a Z_{CNV} value of the number of unique sequences in the each window of the pregnant woman and then locating chromosomal fragment with copy number variation of the pregnant woman to be tested on the basis of the magnitude of the Z_{CNV} value further comprises the steps of:

counting the number of the unique sequences in the each window according to the sequencing depth of each sequence in the sequencing data;

calculating the number of the unique sequences in the each window according to the GC content and the mapping rate of the each chromosome to obtain the pre-correction coverage of the number of the unique sequences in the each window; and

normalizing the pre-correction coverage of the number of the unique sequences in the each window to obtain the Z_{CNV} value of the number of the unique sequences in the each window and determine whether the pregnant woman to be tested has the chromosomal fragment with copy number variation on the basis of the magnitude of the Z_{CNV} value;

if there is a fragment which is 300 Kb or more in length in the sequencing data, and within the fragments which are 300 Kb or more in length, the Z_{CNV} values of the numbers of the unique sequences in 80% or more of the total windows are greater than or equal to 4 or less than or equal to −4, then the fragment which is 300 Kb or more in length is determined to be the fragment with copy number variation from the pregnant woman to be tested.

In above step of normalizing the pre-correction coverage of the number of the unique sequences in the each window to obtain the Z_{CNV} value of the number of the unique sequences in the each window, the normalizing treatment refers to performing (x−u)/sd(x−u) for the corrected value of the number of unique sequences in the each window, wherein x is the corrected value, and u is the mean value of x, sd is the standard deviation. In above step of detecting the fragment with copy number variation of the pregnant woman, by setting the condition of “at least 300 Kb, and Z_{CNV} values in more than 80% of the total windows are greater than or equal to 4 or less than or equal to −4”, a reliable copy number variation fragment of the pregnant woman can be detected by above detection steps of the present application. By correcting the Z_{CNV} value of the chromosome it occurs through the fragment with copy number variation, the false negative resulted by error detection of the fragment with copy number variation of the pregnant woman can be avoided.

In above method of the present application, for the step of performing a Z-test for the each chromosome by using the corrected coverage of the each chromosome to obtain the Zₐₙₑᵤ value, the Zₐₙₑᵤ value is calculated as:

$${Z}_{aneu} = \frac{{x}^{′} - \overline{x}}{s}$$

wherein x̅ represents the pre-correction coverage obtained by the known negative sample population according to a LOESS algorithm, s represents the standard error of (x′−x̅) in the negative sample population. Through the corrected Zₐₙₑᵤ value calculated by above formula can indicate the chromosome aneuploidy more accurately, which will bring a more accurate result.

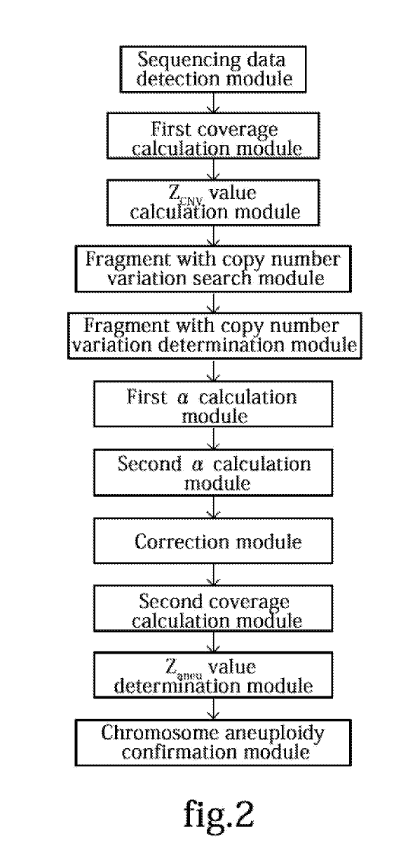

In another exemplary embodiment of the present application, provided is an apparatus for detecting chromosome aneuploidy, as shown in FIG. 2, comprising the following modules:

a sequencing data detection module: for high-throughput sequencing the peripheral blood cell-free DNA from a pregnant woman to produce the sequencing data comprising all the chromosomes;

a first coverage calculation module: for calculating coverage statistics of all of the chromosomes with the sequencing data by segmenting chromosomes into windows so as to produce a pre-correction coverage for each chromosome;

a Z_{CNV} value calculation module: for calculating the Z_{CNV} value on the number of unique sequences in each window from the pregnant woman;

a fragment with copy number variation search module: for searching the fragment that is 300 Kb or more in length in the sequencing data and which has the Z_{CNV} values of the chromosome fragments greater than or equal to 4 or less than or equal to −4 in 80% or more of the total windows;

a fragment with copy number variation determination module: for determining a fragment in the sequencing data that is 300 Kb or more in length and which has Z_{CNV} values of the chromosome fragments greater than or equal to 4 or less than or equal to −4 in 80% or more of the total windows as the fragment with copy number variation of the pregnant woman;

a first α calculation module: for calculating the parameter α according to the formula (1) in the case where the fetus inherits the fragment with copy number variation from the mother, wherein the parameter α represents the impact of the fragment with copy number variation of the pregnant woman on the pre-correction coverage of the each chromosome; m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman;

$$α = \frac{( m - n ) \cdot 2 + n \cdot cn}{m \cdot 2} \qquad ( 1 )$$

a second α calculation module: for calculating the parameter α according to formula (2) in the case where the fetus does not inherit the fragment with copy number variation from the mother, wherein the parameter α is calculated according to formula (2)

$$α = \frac{( m - n ) \cdot 2 + f \cdot n \cdot 2 + ( 1 - f ) \cdot n \cdot cn}{m \cdot 2} \qquad ( 2 )$$

in formula (2), m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;

a correction module: for correcting the pre-correction coverage of the each chromosome by using:

$${x}^{′} = \frac{\hat{x}}{α} ,$$

to produce the corrected coverage of the each chromosome; wherein \hat{x} represents the pre-correction coverage of the each chromosome and x′ represents the corrected coverage of the each chromosome;

a second coverage calculation module: for calculating the Zₐₙₑᵤ value of the each chromosome by using the corrected coverage of the each chromosome;

Zₐₙₑᵤ value determination module: for determining whether the absolute Zₐₙₑᵤ value is greater than or equal to 3;

a chromosome aneuploidy confirmation module: for confirming the chromosome has aneuploidy in the case where the absolute Zₐₙₑᵤ value is greater than or equal to 3.

In the above-described apparatus of the present application, by adding a copy number variation fragment search module, a copy number variation fragment determination module and a correction module, and through screening a region that is at least 300 Kb and has a Z_{CNV} values greater than or equal to 4 or less than or equal to −4 in not less than 80% of the windows on the chromosome of the mother, the fragment with copy number variation of the pregnant woman can be detected by the apparatus of the present application in a more reliable way. In addition, by correcting the Z-test value of the chromosome it occurs through the fragment with copy number variation, the false negative resulted by error detection of the fragment with copy number variation of the pregnant woman can be avoided. By correcting the impact of the fragment with copy number variation on the calculated coverage of the each chromosome, the chromosome aneuploidy confirmation module of the present application can confirm the chromosome aneuploidy in a more accurate way. In the correction module of above apparatus of the present application, the fetal concentration in the calculation formula of parameter α is calculated by the conventional method in the art as described before, which will not be repeated here.

It should be noted that the above-described modules of the present application can be operated as a part of the apparatus in a computing terminal, and the technical solutions achieved by the sequencing data detection module, the first coverage calculation module, the unique sequence calculation module, the fragment with copy number variation search module, the fragment with copy number variation determination module, the first α calculation module, the second α calculation module, the correction module, the second coverage calculation module and the chromosome aneuploidy confirmation module can be executed through using the operator provided by the computing terminal. It is clear that the computing terminal is the hardware apparatus and the operator is also the hardware for executing the program. In addition, the each above mentioned sub-module of the present application can run in a computing device such as the mobile terminal, computer terminal and the like, or can be stored as a part of the storage media.

In the above-described apparatus of the present application, the first coverage calculation module may be obtained by appropriate adjustment according to the difference of sequencing data on the basis of the conventional computing module in the art. In a preferred embodiment of the present application, the first coverage calculation module comprises:

a chromosome window segmentation sub-module: for segmenting all of the chromosomes in the sequencing data into windows with equal size;

a first coverage calculation sub-module: for calculating the coverage statistics in the form of windows with equal size to produce the pre-correction coverage of each chromosome.

Through the calculation in the form of segmented windows with equal size by the first coverage calculation module including the chromosome window segmentation sub-module and the first coverage calculation sub-module, a relatively more robust coverage can be obtained.

In a more preferred embodiment of the present application, the length of each window in the chromosome window segmentation sub-module is 100 Kb, and the overlapping ratio between two adjacent windows is 50%. The calculation module which performs the calculation by segmenting the each window into the size of 100 Kb is advantageous in obtaining a relatively more accurate coverage. In the other hand, by increasing the overlapping ratio between windows, the accuracy for the detection of the fragment with copy number variation can be increased so as to increase the detection efficiency of the fragment with copy number variation of the pregnant woman.

In the above-described apparatus of the present application, a unique sequence calculation module may be obtained by using a conventional calculation module. In a preferred embodiment of the present application, the unique sequence calculation module further comprises:

a unique sequence counting unit: for counting the number of the unique sequences in the each window according to the sequencing depth of each sequence in the sequencing data;

a unique sequence coverage calculation unit: for calculating the number of the unique sequences in the each window according to the GC content and the mapping rate of the each chromosome to obtain the pre-correction coverage of the number of the unique sequences in the each window; and

a unique sequence Z_{CNV} value calculation unit: for normalizing the pre-correction coverage of the number of the unique sequences in the each window to obtain the Z_{CNV} value of the number of the unique sequences in the each window.

In above unique sequence calculation module of the present application, first, according to the sequencing depth of each sequence in the sequencing data, the number of the unique sequences in the each window is counted by running the unique sequence counting unit, and then unique sequence coverage calculation unit is executed according to the GC content and the mapping rate of the each chromosome to calculate the number of the unique sequence of the each window to obtain the pre-correction coverage of the number of the unique sequences in the each window, and then the unique sequence Z_{CNV} value calculation unit is operated to normalize the pre-correction coverage of the number of the unique sequences in the each window to obtain the Z_{CNV} value of the number of the unique sequences in the each window. Above units can be adjusted based on the conventional computing units in the art, which are the foundations and prerequisites for the searching of the fragment with copy number variation search module and as well as the confirmation of the chromosome aneuploidy confirmation module, which provide basis for accurately determining the fragment with copy number variation in the DNA of the mother in the sample to be tested.

In the above-described apparatus of the present application, in the second coverage calculation module, the Zₐₙₑᵤ value is calculated as:

$${Z}_{aneu} = \frac{{x}^{′} - \overline{x}}{s}$$

wherein x̅ is the pre-correction coverage obtained by the known negative sample population according to a LOESS algorithm; s represents the standard error of (x′−x̅) in the negative sample population. The corrected Zₐₙₑᵤ value calculated by above formula can more accurately reflect the aneuploidy of the chromosome, making the detection result more accurate.

In yet another exemplary embodiment of the present application, provided is a kit for detecting chromosome aneuploidy, the kit comprising:

the detection reagents and a detection device: for high-throughput sequencing the peripheral blood cell-free DNA from a pregnant woman to be tested to produce the sequencing data containing all the chromosomes;

a first coverage calculation device: for calculating coverage statistics of all of the chromosomes with the sequencing data by segmenting the chromosomes into windows so as to produce a pre-correction coverage for each chromosome;

a Z_{CNV} value calculation device: for performing a Z-test on the number of unique sequences in each window of the pregnant woman to be tested to obtain the Z_{CNV} value;

a fragment with copy number variation search device: for searching the fragment in the sequencing data that is 300 Kb or more in length and which has the Z_{CNV} values of the chromosome fragments greater than or equal to 4 or less than or equal to −4 in not less than 80% or more of the total windows;

a fragment with copy number variation determination device: for obtaining the fragment with copy number variation of the pregnant woman to be tested on the basis of the magnitude of the Z_{CNV} value;

a first α calculation device: for calculating the parameter α according to the formula (1) in the case where the fetus inherits the fragment with copy number variation from the mother, wherein the parameter α represents the impact of the fragment with copy number variation of the pregnant woman to be tested on the pre-correction coverage of the each chromosome;

$$α = \frac{( m - n ) \cdot 2 + n \cdot cn}{m \cdot 2} \qquad ( 1 )$$

m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman;

a second α calculation device: for calculating the parameter α according to formula (2) in the case where the fetus does not inherit the fragment with copy number variation from the mother, wherein the parameter α is calculated according to formula (2)

$$α = \frac{( m - n ) \cdot 2 + f \cdot n \cdot 2 + ( 1 - f ) \cdot n \cdot cn}{m \cdot 2} \qquad ( 2 )$$

m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;

a correction device: for correcting the pre-correction coverage of the each chromosome by using:

$${x}^{′} = \frac{\hat{x}}{α} ,$$

to produce the corrected coverage of the each chromosome; wherein \hat{x} represents the pre-correction coverage of the each chromosome and x′ represents the corrected coverage of the each chromosome;

a second coverage calculation device: for calculating the Zₐₙₑᵤ value of the each chromosome by using the corrected coverage of the each chromosome;

Zₐₙₑᵤ value determination device: for determining whether the absolute Zₐₙₑᵤ value is greater than or equal to 3;

a chromosome aneuploidy confirmation device: for confirming the chromosome has aneuploidy in the case where the absolute Zₐₙₑᵤ value is greater than or equal to 3.

In the kit of the present application, by adding a fragment with copy number variation search device, a fragment with copy number variation determination device and a correction device, and through screening a region that is at least 300 Kb and which has Z_{CNV} values greater than or equal to 4 or less than or equal to −4 in 80% or more of the total windows on the chromosome of the mother, the fragment with copy number variation of the pregnant woman can be detected by the kit of the present application in a more reliable way. In addition, by correcting the Z_{CNV} value of the chromosome it occurs through the fragment with copy number variation, the false negative resulted by error detection of the fragment with copy number variation of the pregnant woman can be avoided. By correcting the impact of the fragment with copy number variation on the calculated coverage of the each chromosome, the chromosome aneuploidy confirmation device of the present application can confirm the chromosome aneuploidy in a more accurate way. In the correction device of above kit of the present application, the fetal concentration in the calculation formula of parameter α is calculated by the conventional method in the art as described before, which will not be repeated here.

It should be noted that the above-described devices of the present application can be operated as a part of the apparatus in a computing terminal, and the technical solutions achieved by the sequencing data detection device, the first coverage calculation device, the unique sequence calculation device, the fragment with copy number variation search device, the fragment with copy number variation determination device, the first α calculation device, the second α calculation device, the correction device, the second coverage calculation device and the chromosome aneuploidy confirmation device can be executed through using the operator provided by the computing terminal. It is clear that the computing terminal is the hardware apparatus and the operator is also the hardware for executing the program. In addition, each above mentioned sub-device of the present application can run in a computing device such as the mobile terminal, computer terminal and the like, or can be stored as a part of the storage media.

In the above-described kit of the present application, the first coverage calculation device may be obtained by appropriate adjustment according to the difference of sequencing data on the basis of the conventional computing device in the art. In a preferred embodiment of the present application, the first coverage calculation device includes

a chromosome window segmentation component: for segmenting all of the chromosomes in the sequencing data into windows with equal size;

a first coverage calculation component: for calculating the coverage statistics in the form of windows with equal size to produce the pre-correction coverage of the each chromosome.

Through the calculation in the form of segmented windows with equal size by the first coverage calculation device including the chromosome window segmentation component and the first coverage calculation component, a relatively more robust coverage can be obtained.

In a more preferred embodiment of the present application, the size of the each window in the chromosome window segmentation component is 100 Kb, and the overlapping ratio between two adjacent windows is 50%. The calculation device which performs the calculation by segmenting each window into the size of 100 Kb is advantageous in obtaining a relatively more accurate coverage. In the other hand, by increasing the overlapping ratio between windows, the accuracy for the detection of the fragment with copy number variation can be increased so as to increase the detection efficiency of the fragment with copy number variation of the pregnant woman.

In the above-described kit of the present application, a unique sequence calculation device may be obtained by using a conventional calculation device. In a preferred embodiment of the present application, the sequence Z_{CNV} value calculation device further includes:

a unique sequence counting component: for counting the number of the unique sequences in the each window according to the sequencing depth of the each sequence in the sequencing data;

a unique sequence overage calculation component: for calculating the number of the unique sequences in the each window according to the GC content and the mapping rate of the each chromosome to obtain the pre-correction coverage of the number of the unique sequences in the each window; and

a unique sequence Z_{CNV} value calculation component: for normalizing the pre-correction coverage of the number of the unique sequences in the each window to obtain the Z_{CNV} value of the number of the unique sequences in the each window.

In above unique sequence calculation device of the present application, first, according to the sequencing depth of the each sequence in the sequencing data, the number of the unique sequences in each window is counted by running the unique sequence counting unit, and then unique sequence coverage calculation unit is executed according to the GC content and the mapping rate of each chromosome to obtain the pre-correction coverage of the number of the unique sequences in the each window, and then the unique sequence Z_{CNV} value calculation sub-unit is operated to normalize the pre-correction coverage of the number of the unique sequences in the each window to obtain the Z_{CNV} value of the number of the unique sequences in each window. Above units can be adjusted based on the conventional computing units in the art, which are the foundations and prerequisites for the searching of the fragment with copy number variation search device and as well as the confirmation of the chromosome aneuploidy confirmation device, which provide basis for accurately determining the fragment with copy number variation in the DNA of the mother of the to be tested samples.

In the above-described kit of the present application, in the second coverage calculation device, the Zₐₙₑᵤ value is calculated as:

$${Z}_{aneu} = \frac{{x}^{′} - \overline{x}}{s}$$

wherein x̅ is the pre-correction coverage obtained by the known negative sample population according to a LOESS algorithm; s represents the standard error of (x′−x̅) in the negative sample population. The corrected Zₐₙₑᵤ value calculated by above formula can more accurately reflect the aneuploidy of the chromosome, making the detection result more accurate.

The beneficial impacts of the present application will be further described below in combination with specific examples.

### EXAMPLES

### Example 1

In order to test the impact of the correction of the fragment with copy number variations of the pregnant woman on the correction of the chromosome aneuploidy, this example generated a set of simulated data for a to be tested pregnant woman based on the Poisson distribution. In this simulated data, a quantitative copy number of abnormal fragments were added to chromosome 13, 18 and 21, respectively, and the sizes of those copy number variation fragments are from 0.5 Mb to 5 Mb, wherein the step length is 0.25 Mb. Then 3 different concentrations (5%, 10%, 15%) of DNA from normal people were mixed into the simulated data containing the fragment with copy number variations. The whole process is to mimic the impact of the size of different copy number variation fragments on the coverage of chromosome 13, 18 and 21 under different fetal concentrations, and to further test the corrected impact of the fragment with copy number variation of the pregnant woman on the detection of the chromosome aneuploidy. All of the calculations were performed under the assumption that the fetus does not inherent the fragment with copy number variation of the pregnant woman.

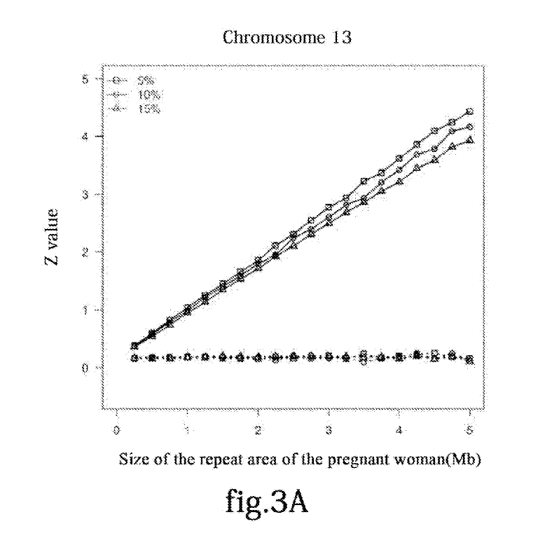

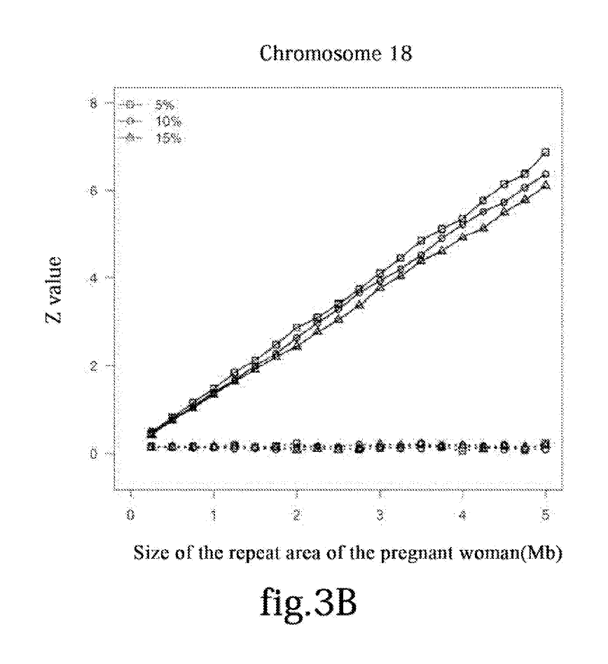

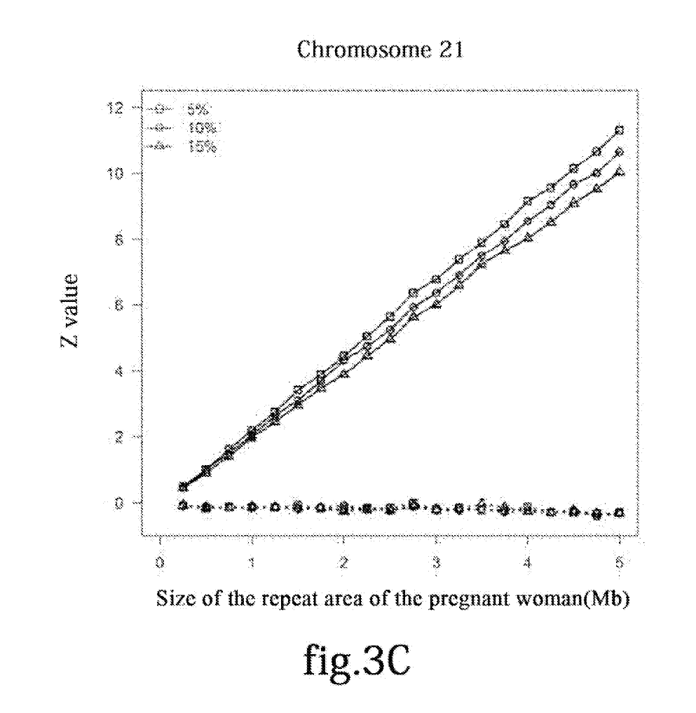

The results of the test are shown in FIGS. 3A, 3B and 3C. In above three figures, the abscissas represents the sizes of the fragment with copy number variations of the pregnant woman where the sample came from, and the ordinates represents the Z values of the chromosomes of this sample. The solid line in the figure shows the Z values of the chromosomes before correction, and the dotted line shows the Z values calculated by the coverage of the chromosomes after the correction through the fragment with copy number variation of the pregnant woman, i.e. Zₐₙₑᵤ value. Square, round and triangular indicates 5%, 10% and 15% fetal concentrations in the samples, respectively.

As can be clearly seen from FIGS. 3A, 3B and 3C, when the Z value was calculated directly with the chromosome coverage, the Z value of the sample increased as the size of the fragment with copy number variation of the pregnant woman increased. In the case of chromosome 21, for example, at 10% fetal concentration, if there is a 3 Mb repeat on chromosome 21 of the pregnant woman, even the fetus does not have 21 trisomy syndrome, the Z value calculated by the previous coverage will be more than 3, which will be determined as a positive. However, as shown by the dotted line, the Z value calculated by the corrected coverage through the method of the present application, i.e. Zₐₙₑᵤ value, were all around the baseline 0, which means that the method of the present application for detecting the chromosome aneuploidy corrected by utilizing the fragment with copy number variation of the pregnant woman is extremely effective.

In order to further verify the effects of the method, the apparatus and the kit provided on the detection of the chromosome aneuploidy in real patients' samples, the following samples from the patients were detected through the method, the apparatus and the kit of the present application as further described in Example 2 and Example 3.

### Example 2

High-throughput sequencing was performed for peripheral blood cell-free DNA from 6615 pregnant women to be tested to produce the sequencing data comprising all of the chromosomes in the samples.

The number of the unique sequences in each window was counted according to the depth of sequencing for each of the sequences in the sequencing data; and the number of the unique sequences in each window was corrected according to the GC content and the mapping rate of each chromosome to produce the corrected coverage of the number of the unique sequences in each window; and the pre-correction coverage of the number of the unique sequences in the each window was normalized to produce the Z_{CNV} value of the number of the unique sequences in each window and to determine whether the pregnant woman possesses the fragment with copy number variation on the basis of the magnitude of the Z_{CNV} value; when there is a fragment 300 Kb or more in the sequencing data, and for the fragment 300 Kb or more, the Z_{CNV} value of the number of the unique sequences in 80% or more of the windows is greater than or equal to 4 or less than or equal to −4, the fragment 300 Kb or more is determined to be the fragment with copy number variation of the pregnant woman.

By utilizing the impact of the fragment with copy number variation of the pregnant woman on the pre-correction coverage of each chromosome, i.e. parameter α, the pre-correction coverage was correct by using

$${x}^{′} = \frac{\hat{x}}{α} ,$$

to produce the corrected coverage of the each chromosome; wherein \hat{x} represents the pre-correction coverage of each chromosome and x′ represents the corrected coverage of each chromosome, and impact of the fragment with copy number variation of the pregnant woman on the pre-correction coverage of each chromosome parameter α was calculated by formula (1) or (2);

The Zₐₙₑᵤ value was calculated by using the corrected coverage of each chromosome according to formula:

$${Z}_{aneu} = \frac{{x}^{′} - \overline{x}}{s}$$

and the aneuploidy of the chromosome was determined based on whether the absolute value of Zₐₙₑᵤ is greater than or equal to 3; wherein when the absolute value of Zₐₙₑᵤ is greater than or equal to 3, the chromosome has aneuploidy, and when the absolute value of Zₐₙₑᵤ is less than or equal to 3, the chromosome does not have aneuploidy.

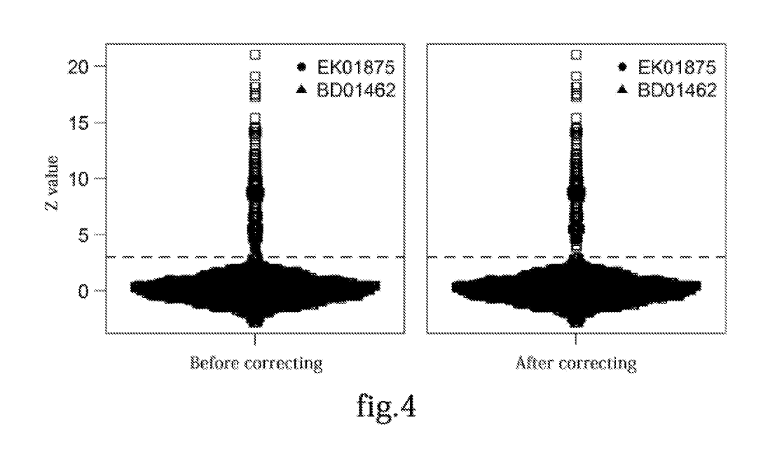

Through above detection method of the present application, it was found that copy number variation fragments of the pregnant woman exist on chromosome 21 of sample EK01875 and BD01462, and the positive results of those two samples were corrected into negative results as shown in FIG. 4 in detail.

The left panel of FIG. 4 (see the figure with color) shows the statistical Z value of chromosome 21 of all the samples detected by the detection methods in the art. As can be seen, the Z values of the negative samples are almost all less than 3 which are close to a normal distribution. The round in the figure indicates sample EK01875 with a Z value of 4.66. The triangle indicates sample BD01462 with a Z value of 3.87.

The right panel of FIG. 4 shows the statistical Z value of chromosome 21 obtained by the detection method of the present application, wherein the sample EK01875 has Zₐₙₑᵤ=2.36, and the sample BD01462 has Zₐₙₑᵤ=1.83.

### Example 3

Above sample (EK01875, 29 years old pregnant woman at about 18 w pregnancy) was detected by the detection apparatus of the present application for chromosome aneuploidy, wherein the apparatus includes:

a sequencing data detecting module: for high-throughput sequencing the peripheral blood cell-free DNA of a pregnant woman to produce sequencing data comprising all the chromosomes;

a first coverage calculation module: for calculating a coverage statistics of all chromosomes in the sequencing data by segmenting into windows so as to produce a pre-correction coverage for each chromosome;

a Z_{CNV} value calculation module: for calculating the Z_{CNV} value on the number of unique sequences in the each of the windows of the pregnant woman;

a fragment with copy number variation search module: for searching the fragment in the sequencing data that is 300 Kb or more e in length in the sequencing data and which has the Z_{CNV} values of the chromosome fragments greater than or equal to 4 or less than or equal to −4 in 80% or more of the total windows;

a fragment with copy number variation determination module: for determining a fragment in the sequencing data that is 300 Kb or more and which has Z_{CNV} values of the chromosome fragments greater than or equal to 4 or less than or equal to −4 in 80% or more of the total windows as the fragment with copy number variation of the pregnant woman;

a first α calculating module: for calculating the parameter α according to the formula (1) in the case where the fetus inherits the fragment with copy number variation from the mother;

a second α calculating module: for calculating the parameter α according to the formula (2) in the case where the fetus does not inherit the fragment with copy number variation from the mother;

a correcting module: for correcting the pre-correction coverage of the each chromosome by using

$${x}^{′} = \frac{\hat{x}}{α}$$

to produce the corrected coverage of the each chromosome;

a second coverage calculating module: for calculating the Zₐₙₑᵤ value of the each chromosome by using the corrected coverage of the each chromosome;

Zₐₙₑᵤ value determination module: for determining whether the Zₐₙₑᵤ value is greater than or equal to 3;

a chromosome aneuploidy confirming module: for confirming the chromosome has aneuploidy in the case where the Zₐₙₑᵤ value is greater than or equal to 3.

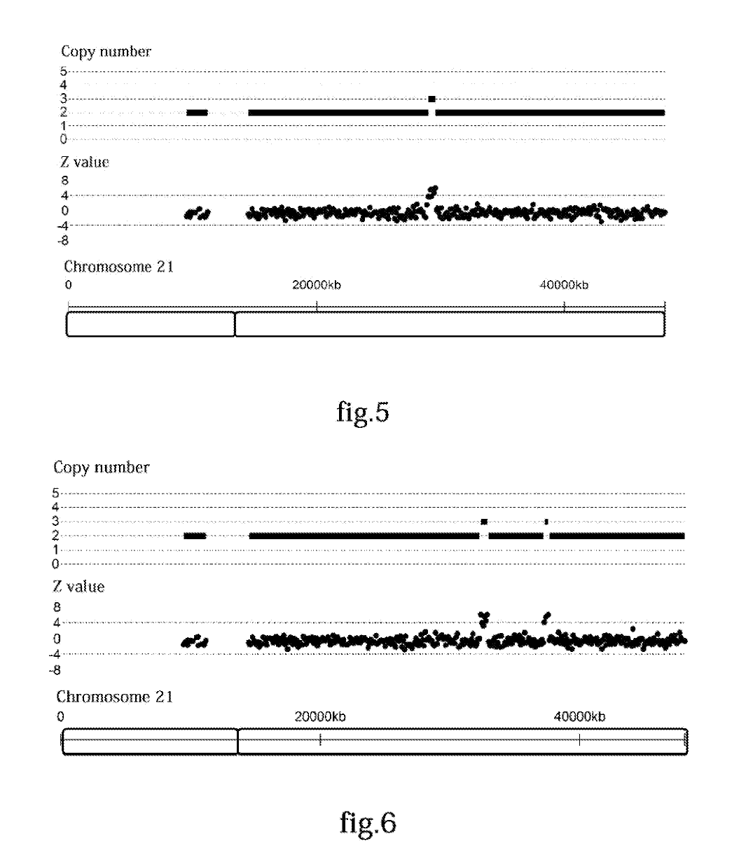

After analyzing the detection of chromosome aneuploidy by using above apparatus of the present application, an 850 kb repeat was found on chromosome 21 of the pregnant woman. As seen in FIG. 5, the regions with repeated copies are 21q22.11 (32361194 bp-32861193 bp) of 500 kb and 21q22.12 (37261194 bp-37611193 bp) of 350 kb, respectively, and their copy numbers are both 3.

Then, the result of the fragment with copy number variations of the pregnant woman was further verified by the Affymetrix CytoScan 750 k SNP chip in the art. Similarly, repeats were detected in regions of 21 q22.11 (32399114 bp-32811202 bp) and 21q22.12 (37292432 bp˜37602701 bp), and the copy numbers are both 3.

It can be seen that the positions detected by the chip are almost 100% identical to the positions detected by the apparatus of the present application. According to the apparatus of the present application, the impact of the fragment with copy number variations of the pregnant woman on the calculation of the coverage of the chromosome, i.e. parameter α, was 1.012, which corrected the Z value characterizing the aneuploidy of the chromosome from 4.66 to 2.36, thereby the result is corrected into negative.

### Example 4

Above sample (BD01462, 24 years old pregnant woman at about 24 w pregnancy) was detected by the kit of the present application for chromosome aneuploidy, wherein the kit comprises:

the detecting reagents and a detecting device: for high-throughput sequencing the peripheral blood cell-free DNA of a pregnant woman to be tested to produce the sequencing data containing all chromosomes;

a first coverage calculation device: for calculating coverage statistics for all of the chromosomes in the sequencing data by segmenting into windows so as to produce a pre-correction coverage for each chromosome;

a unique sequence calculation device: for calculating the Z_{CNV} value of the number of unique sequences in the each window of the to be tested pregnant wonman;

a fragment with copy number variation search device: for searching the fragment in the sequencing data that is 300 Kb or more and which has the Z_{CNV} values of the chromosome fragments greater than or equal to 4 or less than or equal to −4 in 80% or more of the total windows;

a fragment with copy number variation determination device: for obtaining the fragment with copy number variation of the pregnant woman to be tested on the basis of the magnitude of the Z_{CNV} value;

a first α calculation device: for calculating the parameter α according to the formula (1) in the case where the fetus inherits the fragment with copy number variation from the mother;

a second α calculation device: for calculating the parameter α according to the formula (2) in the case where the fetus does not inherit the fragment with copy number variation from the mother;

a correcting device: for correcting the pre-correction coverage of the each chromosome by using

$${x}^{′} = \frac{\hat{x}}{α}$$

to produce the corrected coverage of the each chromosome;

a second coverage calculation device: for calculating the Zₐₙₑᵤ value of the each chromosome by using the corrected coverage of the each chromosome;

Zₐₙₑᵤ value determination device: for determining whether the Zₐₙₑᵤ value is greater than or equal to 3;

a chromosome aneuploidy confirming device: for confirming the chromosome has aneuploidy in the case where the Zₐₙₑᵤ value is greater than or equal to 3.

After analyzing the detection by above kit of the present application, as shown in FIG. 6, a total of 700 kb repeat was found on chromosome 21 of the pregnant woman in region 21q23.1 (28911194 bp˜29611930), and the copy number is 3.

Similarly, 21q21.3 (28973792 bp˜29542400) repeat was found by using Affymetrix CytoScan 750 k SNP chip.

Although the detected copy number is 4 which is slightly different from that of the present application, the position in the result is almost 100% identical to that detected by the kit of the present application, showing the accuracy of the detection method of the present application. According to the kit of the present application, the impact of the fragment with copy number variation of the pregnant woman on the coverage of the chromosome, i.e. parameter α, was 1.009, which corrected the Z value characterizing the aneuploidy of the chromosome from 3.87 to 1.83, thereby correcting the result into negative.

As can be seen from above description, above examples of the present application have achieved the following technical effects: when considering the influence of the fragment with copy number variation of the pregnant woman herself on the calculation of chromosome aneuploidy, the idea of removing the fragment with copy number variation of the mother from the sequencing data is abandoned, and the effect of the fragment with copy number variation with the certain size of the mother on calculating the chromosome aneuploidy is inventively represented by parameter α, which is further used to correct the coverage of each chromosome so as to decrease the influence of the fragment with copy number variation on the determination of the chromosome aneuploidy. The presence of the fragment with copy number variation is not ignored, resulting in a more accurate result for chromosome aneuploidy detected by the method of the present application.

The method, apparatus, or kit of the present application provides a novel detection manner for NIPT of fetus chromosome aneuploidy without any interference from the fragment with copy number variation of the pregnant woman, which improves the accuracy of detection and is suitable for large-scale use.

It will be apparent to those skilled in the art that some of the modules, elements, or steps of the present application described above may be implemented by general computing apparatus, and they can be integrated into one computing apparatus or distributed into a net composed of multiple computing apparatus. Optionally, they can be achieved by program code implementable by the computing apparatus so that they can be stored in a storage apparatus and executed by the computing apparatus. Or multiple modules or step among those can be made into individual integrated circuit modules. In this way, the present application will not be limited by any particular hardware or software.

The foregoing is merely preferred examples of the present application but is not intended to limit the scope of the application. Alterations and variations can be made by the skilled person in the art. Any modifications, equivalent substitutions, improvements, and the like within the spirit and principles of this application are intended to be included within the scope of the present application.

What is claimed is:

1. A method for detecting chromosome aneuploidy, which is characterized in that includes the following steps of:
high-throughput sequencing of the peripheral blood cell-free DNA from a pregnant woman to be tested to produce sequencing data comprising all of the chromosomes;
calculating coverage statistics for all of the chromosomes with the sequencing data by segmenting the chromosomes into windows so as to produce a pre-correction coverage for each chromosome;
performing a Z-test on the number of unique sequence in the each window of the pregnant woman to produce a Z_{CNV} value and then locating chromosomal fragment with the copy number variation of the pregnant woman on the basis of the magnitude of the Z_{CNV} value; wherein chromosomal fragment with the copy number variation of the pregnant woman is the one which is 300 Kb or more in length and which has the Z_{CNV} values of the chromosome fragments greater than or equal to 4 or less than or equal to −4 in 80% or more of the total windows within the fragment which is 300 Kb or more in length,
correcting the pre-correction coverage of the each chromosome by utilizing the impact of the fragment with copy number variation of the pregnant woman on the pre-correction coverage of the each chromosome to produce the corrected coverage for the each chromosome; and
performing a Z-test for the each chromosome by using the corrected coverage of the each chromosome to obtain the Zₐₙₑᵤ value, and determining whether the chromosome has an aneuploidy based on whether the absolute value of Zₐₙₑᵤ is greater than or equal to 3; wherein when the absolute value of Zₐₙₑᵤ is greater than or equal to 3, then it is determined that the chromosome has an aneuploidy;
wherein the impact of the fragment with copy number variation of the pregnant woman to be tested on the pre-correction coverage of the each chromosome is represented by a parameter α,
when the fetus inherits the fragment with copy number variation from the mother, the parameter α is calculated as formula (1):
$$α = \frac{( m - n ) \cdot 2 + n \cdot cn}{m \cdot 2} \qquad ( 1 )$$
when the fetus does not inherit the fragment with copy number variation from the mother, the parameter α is calculated as formula (2):
$$α = \frac{( m - n ) \cdot 2 + f \cdot n \cdot 2 + ( 1 - f ) \cdot n \cdot cn}{m \cdot 2} \qquad ( 2 )$$
in formula (1) and formula (2), m represents the effective length of the chromosome in which the fragment with copy number variation occurs, in the unit of Mb; and n represents the length of the fragment with copy number variation of the pregnant woman to be tested, in the unit of Mb; cn represents the copy number of the fragment with copy number variation found in the pregnant woman to be tested;
in formula (2), f represents the concentration of the cell-free fetal DNA existing in the peripheral blood cell-free DNA of the pregnant woman to be tested, and the concentration f of the cell-free fetal DNA is assumed to be less than 50%;
correcting the pre-correction coverage of the each chromosome by using
$${x}^{′} = \frac{\hat{x}}{α} ,$$
wherein \hat{x} represents the pre-correction coverage of the each chromosome and x′ represents the corrected coverage of the each chromosome.

2. The method according to claim 1, wherein the coverage statistics is calculated by segmenting all of the chromosomes in the sequencing data into windows with equal sizes so as to produce the pre-correction coverage of the each chromosome.

3. The method according to claim 2, wherein the length of each window is 100 Kb and the overlapping ratio between two adjacent windows is 50%.

4. The method according to claim 1, wherein the step of performing a Z-test on the number of unique sequences in the each window of the pregnant woman to be tested to produce the Z_{CNV} value and then locating chromosomal fragment with the copy number variation of the pregnant woman to be tested on the basis of the magnitude of the Z_{CNV} value further includes the steps of:
counting the number of the unique sequences in the each window according to the sequencing depth of each sequence in the sequencing data;
calculating the number of the unique sequences in the each window according to the GC content and the mapping rate of the each chromosome to obtain the pre-correction coverage of the number of the unique sequences in the each window; and
normalizing the pre-correction coverage of the number of the unique sequences in the each window to obtain the Z_{CNV} value of the number of the unique sequences in the each window and determining whether the pregnant woman to be tested has the chromosomal fragment with copy number variation on the basis of the magnitude of the Z_{CNV} value;
if there is a fragment which is 300 Kb or more in length in the sequencing data, and within the fragments which are 300 Kb or more in length, the Z_{CNV} values of the numbers of the unique sequences in 80% or more of the total windows are greater than or equal to 4 or less than or equal to −4, then the fragment which is 300 Kb or more in length is determined to be the fragment with copy number variation of the pregnant woman to be tested.

5. The method according to claim 1, for the step of performing a Z-test for the each chromosome by using the corrected coverage of the each chromosome to obtain the Zₐₙₑᵤ value, the Zₐₙₑᵤ value is calculated as:
$${Z}_{aneu} = \frac{{x}^{′} - \overline{x}}{s}$$
wherein x̅ represents the pre-correction coverage obtained by the known negative sample population according to a LOESS algorithm; s represents the standard error of (x′−x̅) in the negative sample population.

6. An apparatus for detecting chromosome aneuploidy, which is characterized in that includes the following modules:
a sequencing data detection module: for high-throughput sequencing the peripheral blood cell-free DNA from a pregnant woman to be tested to produce the sequencing data comprising all of the chromosomes;
a first coverage calculation module: for calculating a coverage statistics of all of the chromosomes with the sequencing data by segmenting the chromosomes into windows so as to produce a pre-correction coverage for each chromosome;
a Z_{CNV} value calculation module: for calculating the Z_{CNV} value on the number of unique sequences in each window of the pregnant woman;
a fragment with copy number variation search module: for searching the fragment that is 300 Kb or more in length in the sequencing data and which has the Z_{CNV} values of the chromosome fragments greater than or equal to 4 or less than or equal to −4 in 80% or more of the total windows;
a fragment with copy number variation determination module: for determining a fragment in the sequencing data that is 300 Kb or more in length and which has Z_{CNV} values of the chromosome fragments greater than or equal to 4 or less than or equal to −4 in 80% or more of the total windows as the fragment with copy number variation of the pregnant woman;
a first α calculation module: for calculating the parameter α according to the formula (1) in the case where the fetus inherits the fragment with copy number variation from the mother, wherein the parameter α represents the impact of the fragment with copy number variation of the pregnant woman on the pre-correction coverage of the each chromosome;
$$α = \frac{( m - n ) \cdot 2 + n \cdot cn}{m \cdot 2} \qquad ( 1 )$$
in formula (1), m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman;
a second α calculation module: for calculating the parameter α according to formula (2) in the case where the fetus does not inherit the fragment with copy number variation from the mother, wherein the parameter α is calculated according to formula (2)
$$α = \frac{( m - n ) \cdot 2 + f \cdot n \cdot 2 + ( 1 - f ) \cdot n \cdot cn}{m \cdot 2} \qquad ( 2 )$$
in formula (2), m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;
a correction module: for correcting the pre-correction coverage of the each chromosome by using:
$${x}^{′} = \frac{\hat{x}}{α}$$
to produce the corrected coverage of the each chromosome; wherein x̅ represents the pre-correction coverage of the each chromosome and x′ represents the corrected coverage of the each chromosome;
a second coverage calculation module: for calculating the Zₐₙₑᵤ value of the each chromosome by using the corrected coverage of the each chromosome;
Zₐₙₑᵤ value determination module: for determining whether the absolute Zₐₙₑᵤ value is greater than or equal to 3;
a chromosome aneuploidy confirmation module: for confirming the chromosome has aneuploidy in the case where the absolute Zₐₙₑᵤ value is greater than or equal to 3.

7. The apparatus according to claim 6, wherein the first coverage calculation module includes:
a chromosome window segmentation sub-module: for segmenting all of the chromosomes in the sequencing data into windows with equal size;
a first coverage calculation sub-module: for calculating the coverage statistics in the form of windows with equal size to produce the pre-correction coverage of the each chromosome.

8. The apparatus according to claim 7, wherein the length of each window in the chromosome window segmentation sub-module is 100 Kb, and the overlapping ratio between two adjacent windows is 50%.

9. The apparatus according to claim 6, wherein the Z_{CNV} value calculation module includes:
a unique sequence counting unit: for counting the number of the unique sequences in the each window according to the sequencing depth of each sequence in the sequencing data;
a unique sequence coverage calculation unit: for calculating the number of the unique sequences in the each window according to the GC content and the mapping rate of the each chromosome to obtain the pre-correction coverage of the number of the unique sequences in the each window; and
a unique sequence Z_{CNV} value calculation unit: for normalizing the pre-correction coverage of the number of the unique sequences in the each window to obtain the Z_{CNV} value of the number of the unique sequences in the each window.

10. The apparatus according to claim 6, wherein in the second coverage calculation module, the Zₐₙₑᵤ value is calculated as:
$${Z}_{aneu} = \frac{{x}^{′} - \overline{x}}{s}$$
wherein x̅ is the pre-correction coverage obtained by the known negative sample population according to a LOESS algorithm; s represents the standard error of (x′−x̅) in the negative sample population.

11. A kit for detecting the chromosome aneuploidy, which is characterized in that includes:
the detection reagents and a detection device: for high-throughput sequencing the peripheral blood cell-free DNA from a pregnant woman to be tested to produce the sequencing data containing all the chromosomes;
a first coverage calculation device: for calculating a coverage statistics of all of the chromosomes with the sequencing data by segmenting the chromosomes into windows so as to produce a pre-correction coverage for each chromosome;
a Z_{CNV} value calculation device: for performing a Z-test on the number of unique sequences in each window of the pregnant woman to be tested to obtain the Z_{CNV} value;
a fragment with copy number variation search device: for searching the fragment in the sequencing data that is 300 Kb or more in length and which has the Z_{CNV} values of the chromosome fragments greater than or equal to 4 or less than or equal to −4 in 80% or more of the total windows;
a fragment with copy number variation determination device: for obtaining the fragment with copy number variation of the pregnant woman to be tested on the basis of the magnitude of the Z_{CNV} value;
a first α calculation device: for calculating the parameter α according to the formula (1) in the case where the fetus inherits the fragment with copy number variation from the mother, wherein the parameter α represents the impact of the fragment with copy number variation of the pregnant woman to be tested on the pre-correction coverage of the each chromosome;
$$α = \frac{( m - n ) \cdot 2 + n \cdot cn}{m \cdot 2} \qquad ( 1 )$$
m represents the effective length of the chromosome in which the fragment with copy number variation occurs, in the unit of Mb; and n represents the length of the fragment with copy number variation of the pregnant woman to be tested, in the unit of Mb; cn represents the copy number of the fragment with copy number variation found in the pregnant woman;
a second α calculation device: for calculating the parameter α according to formula (2) in the case where the fetus does not inherit the fragment with copy number variation from the mother, wherein the parameter α is calculated according to formula (2)
$$α = \frac{( m - n ) \cdot 2 + f \cdot n \cdot 2 + ( 1 - f ) \cdot n \cdot cn}{m \cdot 2} \qquad ( 2 )$$
m represents the effective length of the chromosome in which the fragment with copy number variation occurs, in the unit of Mb; and n represents the length of the fragment with copy number variation of the pregnant woman, in the unit of Mb; cn represents the copy number of the fragment with copy number variation found in the pregnant woman; f represents the concentration of the cell-free fetal DNA existing in the peripheral blood cell-free DNA of the pregnant woman, and the concentration f of the cell-free fetal DNA is assumed to be less than 50%;
a correction device: for correcting the pre-correction coverage of the each chromosome by using:
$${x}^{′} = \frac{\hat{x}}{α}$$
to produce the corrected coverage of the each chromosome; wherein \hat{x} represents the pre-correction coverage of the each chromosome and x′ represents the corrected coverage of the each chromosome;
a second coverage calculation device: for calculating the Zₐₙₑᵤ value of the each chromosome by using the corrected coverage of the each chromosome;
Zₐₙₑᵤ value determination device: for determining whether the absolute Zₐₙₑᵤ value is greater than or equal to 3;
a chromosome aneuploidy confirmation device: for confirming the chromosome has aneuploidy in the case where the absolute Zₐₙₑᵤ value is greater than or equal to 3.

12. The kit according to claim 11, wherein the first coverage calculation device includes:
a chromosome window segmentation component: for segmenting all of the chromosomes in the sequencing data into windows with equal size;
a first coverage calculation component: for calculating the coverage statistics in the form of windows with equal size to produce the pre-correction coverage of the each chromosome.

13. The kit according to claim 12, wherein the length of the each window in the chromosome window segmentation component is 100 Kb, and the overlapping ratio between two adjacent windows is 50%.

14. The kit according to claim 11, wherein the Z_{CNV} value calculation device includes:
a unique sequence counting component: for counting the number of the unique sequences in the each window according to the sequencing depth of each sequence in the sequencing data;
a unique sequence coverage calculation component: for calculating the number of the unique sequences in the each window according to the GC content and the mapping rate of the each chromosome to obtain the pre-correction coverage of the number of the unique sequences in the each window; and
a unique sequence Z_{CNV} value calculation component: for normalizing the pre-correction coverage of the number of the unique sequences in the each window to obtain the Z_{CNV} value of the number of the unique sequences in the each window.

15. The kit according to claim 11, wherein in the second coverage calculation device, the Zₐₙₑᵤ value is calculated as:
$${Z}_{aneu} = \frac{{x}^{′} - \overline{x}}{s}$$
wherein x̅ is the pre-correction coverage obtained by the known negative sample population according to a LOESS algorithm; s represents the standard error of (x′−x̅) in the negative sample population.
